(19) United States
(12) Patent Application Publication
(10) Pub. No.: US 2012/0100067 A1
(43) Pub. Date: Apr. 26, 2012

(54) Solubilisation Method

(75) Inventors: Ursula Fattler, Riehen (CH); Heinrich Haas, Mainz (DE)

(73) Assignee: MEDIGENE AG, Planegg (DE)

(21) Appl. No.: 12/934,713

(22) PCT Filed: Apr. 3, 2009
(86) PCT No.: PCT/EP2009/002485

§ 371 Date: May 9, 2011

(30) Foreign Application Priority Data
Apr. 4, 2008 (EP) 08006874.5

(51) Int. Cl. A61K 9/127; A61K 51/12; A61K 31/475; A61P 35/00; A61K 49/00; A61K 31/337

(52) U.S. Cl. 424/1.11; 424/450; 424/9.1; 514/449; 514/283

(57) ABSTRACT

The present invention relates to the solubilisation of an active agent in a lipid dispersion, in particular to the solubilisation of an active agent in a suspension of preformed empty liposomes.

### INTRODUCTION

The present invention relates to the solubilisation of an active agent in a lipid dispersion, in particular to the solubilisation of an active agent in a suspension of preformed empty liposomes.

In the recent years liposomes have become an important tool in pharmaceutical industry for the delivery of drugs. Liposomes are capable of influencing pharmacokinetics by sustained release of the drug in the body or by reducing side effects by limiting the free concentration of a drug. Hydrophobic drugs are formulated in liposomes by integration into lipid bilayers. Hydrophilic agents may be formulated in liposomes by encapsulation in the aqueous core of liposomes.

In the most common procedures for solubilising an agent in liposomes, like in the “film method” or in the “ethanol/ether injection method”, or variations thereof, the active agent is present during liposome formation. In consequence, liposome and active agent are stored together. This approach is especially unfavorable in cases where liposome and active agent have considerable different stabilities. These methods do not allow a solubilisation of the active agent in the lipid phase directly prior to use, i.e. the later encapsulation of the agent into a preformed liposome, as usually these prior art liposomes require further processing, like sizing or sterile filtration and the like to be suitable to be used as a pharmaceutical.

In certain cases it is desirable to encapsulate different types of active agents in preformed liposomes to be suitable for certain applications like delivery of therapeutic or diagnostic agents. However, encapsulation of an active agent or if desired, of different types of active agents, into preformed liposomes is not possible by prior art methods since they require the presence of the active agent already during liposome formation.

Encapsulation of active agents into preformed liposomes is facilitated by methods using an ion or pH gradient at the liposomal membrane. Such methods have been disclosed by Bally et al. (U.S. Pat. No. 5,077,056) Hope et al. (U.S. Pat. No. 5,785,987), Janoff et al. (U.S. Pat. No. 5,837,279) and Mayer et al. (U.S. Pat. No. 6,083,530). It has been demonstrated by Janoff et al. that the method can be applied to liposomes which had been dehydrated for prolonged storage. However, the method is limited to ionisable agents which have a good solubility in water.

It has been disclosed by Schneider et al. (U.S. Pat. No. 4,229,360) and Deamer et al. (U.S. Pat. No. 4,515,736) that preformed liposomes may also be loaded with active agents without the presence of an ion gradient. In the disclosed method, a suspension of preformed liposomes is mixed with an active agent, and the mixture is subsequently dehydrated. Upon rehydration in a suitable medium, the agent is encapsulated in the liposomes. The method has been further improved by the addition of sugar to the initial mixture of liposomes and active agent prior to dehydration (Gregoriadis et al., EP 1 087 754). However in liposomes prepared according to these methods, the liposome and the active agent can not be stored separately. Also, the method is limited to the encapsulation of agents with a high solubility in aqueous solutions.

A similar process is disclosed by Zadi (EP 1 259 225), wherein the preformed liposomes are mixed with a lipophilic particulate active agent, such as paclitaxel, which has been obtained by precipitation. The agent is encapsulated into the liposomes by dehydration and rehydration of the mixture. Again, this method does not enable a solubilisation of the active agent in the liposomal phase directly prior to use but requires the joint storage of active agent and liposomal phase.

The latter limitation has been overcome by Collins et al., U.S. Pat. No. 6,355,267, for hydrophilic agents (e.g. sugars, soluble proteins). In the respective method liposomes were prepared by the film method and subsequently subjected to freeze-thaw cycles and dehydration. Upon rehydration of the liposomes in a medium comprising a solubilised, hydrophilic active agent, said agent was encapsulated in the liposomes. It is assumed that the method achieves high encapsulation efficiencies due to the large volume of the liposomes. Thus the method is limited to agents with a high solubility in the final aqueous medium. The method is also limited to liposome preparations which can be dehydrated, since the rehydration step is required for the encapsulation of the active agent. Consequently the method is not suitable for liposome preparations which have to be stored in a frozen form.

In view of the current state of the art, the underlying problem of the current invention is the provision of an improved process for the solubilisation of an active agent in a lipid dispersion, such as a liposomal suspension. The process should be particularly suitable for the solubilisation of agents with a poor solubility in an aqueous phase. The desired process should enable solubilisation of an active agent directly prior to use and provide for a separate storage of lipid dispersion and active agent.

### DESCRIPTION OF THE INVENTION

The solution of the above described problem is provided by the current invention as described in the embodiments characterised in the claims and the more detailed description provided by the application.

In a first aspect the invention relates to a process for solubilising at least one active agent in a lipid dispersion comprising at least one lipid, comprising incubating an active agent in an undissolved form with a lipid dispersion. The undissolved form of the active agent may be for example a crystalline form of different morphology and size or a powder form.

In a second aspect, the invention relates to a process for solubilising at least one active agent in a lipid dispersion comprising at least one lipid, comprising the steps of:
i) freezing or dehydrating a primary lipid dispersion comprising an aqueous medium and optionally one or more excipients,
ii) thawing the frozen lipid dispersion or rehydrating the dehydrated lipid dispersion of step i) to obtain a secondary lipid dispersion, and
iii) incubating the secondary lipid dispersion of step ii) with an active agent.

It is a feature of the current invention that the lipid dispersion does not have to be subjected to freezing, thawing, dehydration, rehydration or other physical processes that are used to promote the solubilisation of an active agent in a lipid dispersion after the active agent has been incubated with said lipid dispersion.

According to the invention the two aspects of the invention may be joint in a process for solubilising at least one active agent in a lipid dispersion comprising incubating said active agent in an undissolved form with said lipid dispersion, wherein said lipid dispersion is obtained by freezing/thawing or dehydration/rehydration as described above for the secondary lipid dispersion.

The inventive process is preferably performed with agents that have higher partitioning into the lipid phase compared to the aqueous phase in a system which comprises an aqueous and a lipid phase. Thus, the process preferably relates to the solubilisation of an agent in the lipid phase of the lipid dispersion. Agents that have a higher partitioning into the lipid phase have a positive log P, preferably a log P of greater than 1, more preferably of greater than 3. Preferably the agents are hydrophobic and/or have a low solubility in water.

Preferably, the active agent employed in the inventive process is a small molecule, i. e. with a mole weight of about 1000 Da or less. Preferably, the active agent is therapeutically and/or diagnostically active.

The lipid dispersion employed in the current invention preferably is a colloidal suspension, most preferably a liposomal suspension. Thus the process of solubilisation refers to the loading of an agent into liposomes.

In another aspect, the invention relates to a lipid dispersion comprising at least one active agent obtainable by the inventive process. Preferably, said dispersion is characterised by a release of the active agent from the lipid phase of said dispersion of less than about 6% in at least 3 days.

In a further aspect, the invention relates to the preparation of a pharmaceutical composition comprising the above described processes, and to a pharmaceutical composition obtainable by the above mentioned processes. Thus the invention also relates to the use of a liposomal preparation obtainable by the process described above as a medicament.

In another aspect, the invention provides a kit comprising a frozen or dehydrated lipid dispersion, optionally a rehydration buffer, an instruction manual and optionally a diagnostic or therapeutic agent.

In still another aspect, the invention relates to a method for increasing the solubilisation efficiency of preformed liposomes for an active agent comprising treating preformed liposomes with at least one freezing and thawing and/or dehydrating and rehydrating step prior to solubilising an active agent in said treated liposomes, compared to untreated preformed liposomes.

It has been described in the prior art, for example by Schneider et al. (U.S. Pat. No. 4,229,360), Deamer et al. (U.S. Pat. No. 4,515,736), Gregoriadis et al. (EP 1 087 754), Zadi (EP 1 259 225), or Collins et al. (U.S. Pat. No. 6,355,267), that freezing and thawing or dehydration and rehydration of lipid dispersions can be used to facilitate solubilisation of agents into lipid dispersion. In these processes, however, it is necessary to perform freezing/thawing as well as dehydration/rehydration in the presence of the agent which is to be solubilised. The physical stress induced by the mentioned processes provides a driving force for the solubilisation.

In contrast thereto, in the inventive process, the agent which is to be solubilised is added to the lipid dispersion after freezing/thawing or dehydration/rehydration have been performed. Consequently the agent is not present during the induction of physical stress on the lipid dispersion.

Nonetheless it has been surprisingly found that freezing/thawing or dehydration/rehydration promote the solubilisation of an agent even if none of said processes is performed in the presence of said agent, but if the agent is added to the lipid dispersion even after said processes have already been terminated.

It has been surprisingly found that agents, which are poorly soluble in water can efficiently be solubilised by exposing them to a lipid dispersion in an undissolved form, e.g. in crystalline form or as a powder.

It is well established that poorly soluble, and/or lipophilic agents, can be solubilised by loading them into colloidal lipid dispersions, such as liposomes, and a variety of different procedures is described in the literature (New, R.R.C. Liposomes—Apractical Approach (1990) Oxford University Press, 33-104). However, current processes for solubilising an agent in a lipid preparation include a step where the agent is dispersed into the aqueous phase and/or the lipid matrix, by aid of an external force or by a suitable preparation method. This is in order to improve solubilisation efficacy. Hence, in experimental practice all loading protocols include steps to accelerate solubilisation by an external force. This is due to the much lower solubility in water (in many cases by several orders of magnitude) in relation to the targeted concentration of the solubilised agent. Since the agent must pass through the aqueous phase for solubilisation, the process is very inefficient. If poorly water soluble agents are presented in crystalline or any other undissolved form to an aqueous medium, solubilisation takes a long time to achieve sufficient concentrations in the aqueous medium. For example, the solubility of paclitaxel in water is well below 1 μg/ml, while practically useful preparations of paclitaxel must have concentrations of at least 100 μg/ml. Since the drug must be first dissolved in water for partitioning into the lipid phase, the low solubility in water was a bottleneck which inhibited fast and efficient solubilisation.

It was therefore surprising that, using the methods of the invention, high concentrations of solubilised agents could be obtained within minutes, when they were exposed to the colloidal preparation in crystalline form, even though the agents were poorly soluble in water. According to conventional understanding it would have been expected that solubilisation would take much more time and would have had a low efficacy. For example, in order to determine the maximum solubility of poorly soluble agents in water, shaking for many hours is necessary. Therefore, in all established protocols for solubilisation of poorly soluble agents, external stress is applied (New, 1990; Zadi, EP 1 259 255), for example in order to provide an utmost fine dispersion of the agent in the aqueous phase or to force the agent into the lipid matrix.

It has been further surprisingly found that the solubilising and therewith loading capacity of a lipid dispersion for an active agent could be improved when the lipid dispersion is subjected to at least one freeze/thaw cycle, or dehydration/rehydration cycle, or a combination of such processing steps, prior to incubation with the active agent. The increase of loading capacity refers to the amount of active agent which is solubilised in a lipid dispersion treated according to the current invention compared to an untreated dispersion under identical experimental conditions as described in the examples. This was surprising, because, according to general understanding, the loading capacity of the lipid dispersion should be the same before and after freeze-thawing/dehydration-rehydration. It would have been expected that a treatment like freezing and subsequent thawing and/or dehydration with subsequent rehydration does not affect the solubilisation capacity of the lipid dispersion.

It is well known that lyoptropic lipid phases may depend on the history of preparation, and processes like swelling of lipid multilayers can be rather slow, i.e. typical time scales can be several hours or even several days. However in the present case, the lipids are dispersed to the fully hydrated state (i.e. large excess of water is already present) and also lipid membranes in a fluid-like (liquid crystalline) state showed the described effect. For such systems, no lasting effect of freeze-thawing/dehydration-rehydration on the membrane characteristics, which could be relevant for the solubilising capacity for an agent, was expected. Surprisingly, the changes of loading capacity induced by the described processes are long lasting. It has been observed that the improved loading capacity was maintained for 7, 14, 21 and even 28 days and might be maintained even longer.

Surprisingly it has been found that the increase of loading capacity of freeze/thawed lipid dispersions in comparison to untreated dispersions is much higher in liposome dispersions comprising at least two different types of lipids, such as binary lipid mixtures. Such an effect of the lipid composition could not be predicted, even after the identification of the basic effect of freezing/thawing on the loading of liposomes.

As a consequence, hydrophobic agents having a poor solubility in aqueous media, can be solubilised by colloidal lipid dispersions to a concentration which is higher than the solubility of said agent in said aqueous medium. In one experiment, when paclitaxel was added to an aqueous liposomal suspension in undissolved form, it could be solubilised in said suspension in a concentration of up to 1600 μM, although the solubility of paclitaxel in a corresponding aqueous buffer is well below 1 μM. Thus the drug was loaded into liposomes with a very high efficiency.

Furthermore it has been surprisingly found that an active agent loaded into a freeze/thawed lipid dispersion is released from the lipid phase of said dispersion much slower in comparison to an agent loaded into an untreated lipid dispersion.

The invention enables fast, easy and efficient loading of an agent into a lipid dispersion like a suspension of preformed liposomes. High concentrations of poorly soluble agents in an aqueous environment are obtained. By loading the agent into the lipid particles the release and/or delivery characteristics can be modified.

As described above, currently applied methods for solubilising poorly soluble agents require dispersion of such agents by different means. Therefore it was surprisingly found that with the inventive process high concentrations of agents which are poorly soluble in the aqueous phase could be obtained within time scales as short as few minutes by exposing undissolved (e.g. crystalline) material to colloidal lipid preparations.

The advantages of the described process are:
Active agents which are poorly soluble in water, can be presented in an aqueous phase at high concentrations
The process is very fast, i.e., it takes only few minutes
By the described pre-treatment, the loading capacity of the colloidal dispersion, can be even elevated, i.e., the maximum concentration of solubilised agent can be elevated
No organic solvents or detergents are necessary for solubilisation of the agent by the colloidal lipid dispersion
The method can be used to obtain preparations of active agents for application to a patient.
Preparations can be made directly before application to a patient and preparations which are chemically or physically instable can be applied. Therefore, more types of pharmaceutical preparations can be made available for application to a patient. For example, there are many preparations which are stable only for very limited time, i.e., few hours. Such stability is sufficient for application to a patient if the product is prepared immediately before application, even though regular industrial manufacturing and storage is not possible.
Active agents can be efficiently loaded into a colloidal matrix to affect its pharmacokinetic behavior. For example formulations for drug delivery or controlled release can be prepared.
The stability of an agent in a preformed formulation of a colloidal lipid dispersion can be improved when the lipid dispersion is treated with at least one freezing/thawing and/or dehydration/rehydration step prior to incubating/incorporating the active agent, i.e., by the process a virtually metastable preparation (with regard to drug release from the carrier matrix) can be transformed into a virtually stable one.

The invention possesses several advantages over the present state of the art by loading agents into established lipid dispersions like preformed liposomes. At first, it provides a simple and easy method by which loading of an active agent into lipid dispersion is achieved. The loading efficacy can be improved by the described pre-treatment. In contrast to other methods, the method described herein is based on a simple physical treatment, and does not require the addition of agents like solvents, detergents, or special salts and ionophores to build up a concentration gradient within the lipid particles. Unlike methods that imply the use of a charge gradient, the current invention is not limited to ionisable agents. Also, no addition of ethanol or other solvents, which would be undesired for a subsequent intravenous application, to the suspension is required as described by Hope in U.S. Pat. No. 6,447,800. Also the use of surfactants, which often are undesired because of their potential toxicity, can be avoided.

Solubilisation of an agent in a lipid dispersion according to the present invention can be accomplished within very short time scales (minutes) which makes it easy to prepare and directly use the obtained preparation without substantial further delay of time. Particularly, it is possible to obtain the preparation directly before administration to a patient.

The invention permits to improve the stability of certain types of preparations of poorly soluble agents in a colloidal dispersion of amphiphilic molecules. This is particularly the case, if such a formulation is metastable, and the stability is limited by physical degradation. By the process, the stability and thus the lifetime of such preparations can be improved.

The process is preferably used with agents that are poorly soluble in water and which partition into the lipid phase in lipid dispersions. Therefore, the process is especially advantageous for agents for which higher concentrations in an aqueous phase compared to the solubility of the agent in pure water are needed.

Loading of an active agent into a colloidal lipid dispersion prior to use, i.e. before administration to a subject in need thereof, has advantages over loading an agent during preparation of the lipid dispersion because colloidal lipids and active agent can be stored individually. Often, lipid particles comprising a drug are more difficult to be stabilized than pure empty lipid particles. Thus, lyophilisation (or other types of stabilization like freezing) of the pure lipid component is much easier as of a liposome preparation comprising the drug. Conventional lyophilisation can be avoided, and other conservation processes like spray drying, which are difficult to perform with drug comprising lipid particles because of drug degradation due to thermal or other stress can be used. Also, formulations of lipid particles, which for a given reason must not be lyophilised, can be prepared.

The inventive methods enables separate storage of preformed liposomes and agents to be encapsulated and thus improves formulation of agents which have a short shelf live, and would thus otherwise not be applicable in a liposomal formulation. For example, lipid particles can be loaded with radioactive drugs with short half life for therapeutic or diagnostic applications. Other examples are drugs which are chemically instable, or which need to be produced directly before application by other reasons. Other examples are preparations, where very high concentrations of an active agent are present, which would not be stable enough to provide sufficient shelf life. Thus higher doses of an agent can be applied to a patient.

Furthermore, the invention enables loading of different agents into one and the same preformed liposome species for therapeutic or diagnostic purpose.

The lipid dispersion used in the current invention may comprise lipids or lipid particles in form of liposomes, emulsions, micelles or mixed micelles, oligo- or multilamellar vesicles or other lyotropic phases comprising monomeric or polymeric amphphilic moieties. Preferably, the lipid dispersion comprises lipid bilayers. More preferably, the lipid dispersion is a colloidal dispersion. Most preferably, the lipid dispersion is a liposomal suspension.

The liposomes employed in the inventive method or process may have different sizes, lamellarity and structure. Preferably, the liposomes have an average diameter of about 50 nm to about 500 nm, more preferably of about 100 to about 300. Most preferred is a size of about 100 to about 200 nm. The liposomes may be unilamellar liposomes.

The lipids employed in the current invention may be natural and/or synthetic lipids, lipids comprising different head group moieties, different hydrophobic moieties, in particular regarding chain length and saturation, different charge and different phase state can be applied.

The lipid dispersion employed in the invention may comprise neutral, anionic and/or preferably cationic lipids. Cationic lipids are preferably comprised in an amount of at least about 30 mol %, more preferably of at least 40 mol % and most preferably in an amount of at least 50 mol % of total lipids. The resulting cationic liposomes have a positive zeta potential, preferably greater than about +20 mV, more preferably greater than about 30 mV, and most preferably greater than about 40 mV when measured in about 0.05 mM KCl solution at about pH 7.5.

Neutral or anionic lipids may be selected from sterols or lipids such as cholesterol, phospholipids, lysolipids, lysophospholipids, sphingolipids or pegylated lipids with a neutral or negative net charge. Useful neutral and anionic lipids thereby include: phosphatidylserine, phosphatidylglycerol, phosphatidylinositol (not limited to a specific sugar), fatty acids, sterols, containing a carboxylic acid group for example, cholesterol, 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-diacyl-glycero-3-phosphocholines, 1,2-distearylphosphatidylcholine (DSPC), 1,2-dipalmitylphosphatidylcholine (DPPC), 1,2-dimyristylphosphosphatidylcholine (DMPC), phosphatidylcholine preferably egg PC, soy PC and sphingomyelin. The fatty acids linked to the glycerol backbone are not limited to a specific length or number of double bonds. Phospholipids may also have two different fatty acids. Preferably, the further lipids are in the liquid crystalline state at room temperature and they are miscible (i.e. a uniform phase can be formed and no phase separation or domain formation occurs) with the used cationic lipid, in the ratio as they are applied. In a preferred embodiment the neutral lipid is 1,2-dioleylphosphatidylcholine (DOPC).

Cationic lipids may preferably be selected from a group comprising N-[1-(2,3-dioleoyloxy)propyl]-N,N,N-trimethyl ammonium salts, preferably the chloride or methylsulfate. Preferred representatives of the family of -TAP lipids are DOTAP (dioleoyl-), DMTAP (dimyristoyl-), DPTAP (dipalmitoyl-), or DSTAP (distearoyl-). Other useful lipids for the present invention may include: DDAB, dimethyldioctadecyl ammonium bromide; 1,2-diacyloxy-3-trimethylammonium propanes, (including but not limited to: dioleoyl, dimyristoyl, dilauroyl, dipalmitoyl and distearoyl; also two different acyl chains can be linked to the glycerol backbone); N-[1-(2,3-dioloyloxy)propyl]-N,N-dimethyl amine (DODAP); 1,2-diacyloxy-3-dimethylammonium propanes, (including but not limited to: dioleoyl, dimyristoyl, dilauroyl, dipalmitoyl and distearoyl; also two different acyl chain can be linked to the glycerol backbone); N-[1-(2,3-dioleyloxy)propyl]-N,N,N-trimethylammonium chloride (DOTMA); 1,2-dialkyloxy-3-dimethylammonium propanes, (including but not limited to: dioleyl, dimyristyl, dilauryl, dipalmityl and distearyl; also two different alkyl chain can be linked to the glycerol backbone); dioctadecylamidoglycylspermine (DOGS); 3β-[N-(N′,N′-dimethylaminoethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA); β-alanyl cholesterol; cetyl trimethyl ammonium bromide (CTAB); diC14-amidine; N-tert-butyl-N′-tetradecyl-3-tetradecylamino-propionamidine; 14Dea2; N-(alpha-trimethylammonioacetyl)didodecyl-D-glutamate chloride (TMAG); O,O′-ditetradecanoyl-N-(trimethylammonioacetyl)diethanolamine chloride; 1,3-dioleoyloxy-2-(6-carboxy-spermyl)-propylamide (DOSPER); N,N,N′,N′-tetramethyl-N,N′-bis(2-hydroxylethyl)-2,3-dioleoyloxy-1,4-butanediammonium iodide; 1-[2-(acyloxy)ethylpalkyl(alkenyl)-3-(2-hydroxyethyl)-imidazolinium chloride derivatives as described by Solodin et al. (Solodin et al., 1995), such as 1-[2-(9(Z)-octadecenoyloxy)ethyl]-2-(8(Z)-heptadecenyl-3-(2-hydroxyethyl)imidazolinium chloride (DOTIM), 1-[2-(hexadecanoyloxy)ethyl]-2-pentadecyl-3-(2-hydroxyethyl)imidazolinium chloride (DPTIM), 2,3-dialkyloxypropyl quaternary ammonium derivatives, containing a hydroxyalkyl moiety on the quaternary amine, as described e.g. by Feigner et al. (Feigner et al., 1994) such as: 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), 1,2-disteryloxypropyl-3-dimethyl-hydroxyethyl ammonium bromide (DSRIE); cationic esters of acyl carnitines as reported by Santaniello et al. (U.S. Pat. No. 5,498,633); cationic triesters of phosphatidylcholine, i.e. 1,2-diacyl-sn-glycerol-3-ethylphosphocholines, where the hydrocarbon chains can be saturated or unsaturated and branched or non-branched with a chain length from C12 to C24, the two acyl chains being not necessarily identical.

In a preferred embodiment of the current invention, the lipid dispersion consists of at least two different types of lipids. Preferably, the dispersions comprises the two lipids in a ratio between about 90:10 and 10:90, more preferably between about 75:25 and 25:75, most preferably about 50:50.

Preferably, the lipid dispersion comprises DOTAP and DOPC, in a ratio between about 75:25 and 25:75.

In another preferred embodiment at least one of the lipids, preferably all of the lipids, employed in the invention has a phase transition temperature which is lower than room temperature (23° C.).

In another preferred embodiment at least one of the lipids comprises at least one unsaturated or branched alkyl chain.

In an alternative embodiment of the current invention, the invention employs a colloidal dispersion of an amphiphilic monomeric or polymeric molecule instead of a lipid dispersion. All embodiments of the inventions described for lipid dispersions which are not related to the lipid itself, will also apply to these dispersions of amphiphilic monomeric or polymeric molecules.

In a preferred embodiment of the current invention, the lipid dispersion is a liposomal suspension. Suitable methods for the preparation of liposomes, are known to the person skilled in the art and any method which is suitable to prepare lipid particles may be used to provide lipid particles for the inventive process. In one embodiment of the invention the suspension comprises liposomes which may be prepared according to the “lipid film method” or by “ethanol injection”, which are known to those skilled in the art and are disclosed in WO 2004/002468 for example. Other methods for the preparation of liposomes are described in New et al.

The lipid dispersion employed by the invention comprises an aqueous medium. Besides water, the aqueous medium of the present invention may comprise one or more further liquid constituents which are at least partially miscible with water, preferably an organic solvent, more preferably an alcohol (e.g. a C₁₋₄ alcohol such as methanol, ethanol, propanol, butanol and combinations thereof, etc.) or a ketone (e.g. a C₁₋₄ ketone such as acetone, methylethyl-ketone and combinations thereof, etc.). Furthermore the dispersion may comprise additives such as, but not limited to, pH-stabilising-agents, salts, antioxidants, cryoprotectants or other excipients.

In a preferred embodiment of the present invention, the lipid dispersion comprises at least one excipient. The excipient may be selected from the group comprising water-soluble sugars such as glucose, saccharose, raffinose, galactose, maltose, lactose, mannitol, sorbitol or trehalose, most preferably trehalose. The excipient may also be an alcohol (e.g. a C₁₋₄ alcohol such as methanol, ethanol, propanol, butanol and combinations thereof, etc.), a polymer (e.g. polyethylenglycol, or a polydextran, etc.) or a salt. Preferably, the inventive lipid dispersion comprises an excipient in a concentration of at least 0,1% (m/v), preferably of at least about 1% (m/v), more preferably of at least about 5% (m/v), and most preferably of about 10% (m/v) with respect to the total volume of the preparation. Preferably, all agents or media comprised in the dispersion are pharmaceutically acceptable.

The active agent solubilised in the present invention is therapeutically and/or a diagnostically active.

Agents which are to be solubilised may preferably be agents which are not easy to be effectively delivered for several reasons, such as agents which are poorly soluble in water, sensitive (including thermally labile) active agents, or small molecules. Preferably, the active agent is a small molecule.

In one aspect of the invention, oligo- or polypeptides, proteins, or nucleotides are excluded from species of active agents.

Examples of therapeutically active agents are: Analgesics and anti-inflammatory agents, for example aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac. Anthelmintics, for example albendazole, bephenium hydroxynaphthoate, cambendazole, dichlorophen, ivermectin, mebendazole, oxamniquine, oxfendazole, oxantel embonate, praziquantel, pyrantel embonate, thiabendazole. Anti-arrhythmic agents, for example amiodarone HCl, disopyramide, flecainide acetate, quinidine sulphate. Anti-bacterial agents: benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim. Anti-coagulants, for example dicoumarol, dipyridamole, nicoumalone, phenindione. Anti-depressants, for example amoxapine, maprotiline HCl, mianserin HCl, nortriptyline HCl, trazodone HCl, trimipramine maleate. Anti-diabetics, for example acetohexamide, chlorpropamide, glibenclamide, gliclazide, glipizide, tolazamide, tolbutamide. Anti-epileptics, for example beclamide, carbamazepine, clonazepam, ethotoin, methoin, methsuximide, methylphenobarbitone, oxcarbazepine, paramethadione, phenacemide, phenobarbitone, phenytoin, phensuximide, primidone, sulthiame, valproic acid. Anti-fungal agents, for example amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecenoic acid. Anti-gout agents, for example allopurinol, probenecid, sulphin-pyrazone. Anti-hypertensive agents, for example amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCl, reserpine, terazosin HCl. Anti-malarials, for example amodiaquine, chloroquine, chlorproguanil HCl, halofantrine HCl, mefloquine HCl, proguanil HCl, pyrimethamine, quinine sulphate. Anti-migraine agents, for example dihydroergotamine mesylate, ergotamine tartrate, methysergide maleate, pizotifen maleate, sumatriptan succinate. Anti-muscarinic agents, for example atropine, benzhexol HCl, biperiden, ethopropazine HCl, hyoscyamine, mepenzolate bromide, oxyphencylcimine HCl, tropicamide. Anti-neoplastic agents, for example carmustine, cisplatin, fluorouracil; adriamycin, asparaginase, azacitidine, azathioprine, bleomycin, busulfan, carboplatin, cisplatin, carmustine, chlorambucil, cyclophosphamide, cyclosporine, cytarabine, dacarbazine, dactinomycin, daunorubicin, doxorubicin, estramustine, etoposide, etretinate, filgrastin, floxuridine, fludarabine, fluorouracil, florxymesterone, flutamide, goserelin, hydroxyurea, ifosfamide, leuprolide, levamisole, limustine, nitrogen mustard, melphalan, mercaptopurine, methotrexate, mitomycin, mitotane, pentostatin, pipobroman, plicamycin, procarbazine, sargramostin, streptozocin, tamoxifen, taxanes, teniposide, thioguanine, uracil mustard, vinblastine, vincristine and vindesine. Anti-protazoal agents, for example benznidazole, clioquinol, decoquinate, diiodohydroxyquinoline, diloxanide furoate, dinitolmide, furzolidone, metronidazole, nimorazole, nitrofurazone, ornidazole, tinidazole. Anti-thyroid agents, for example carbimazole, propylthiouracil. Anxiolytic, sedatives, hypnotics and neuroleptics, for example alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone. β-Blockers, for example acebutolol, alprenolol, atenolol, labetalol, metoprolol, nadolol, oxprenolol, pindolol, propranolol. Cardiac inotropic agents, for example amrinone, digitoxin, digoxin, enoximone, lanatoside C, medigoxin. Corticosteroids, for example beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone. Diuretics, for example acetazolamide, amiloride, bendrofluazide, bumetanide, chlorothiazide, chlorthalidone, ethacrynic acid, frusemide, metolazone, spironolactone, triamterene. Anti-parkinsonian agents, for example bromocriptine mesylate, lysuride maleate. Gastro-intestinal agents, for example bisacodyl, cimetidine, cisapride, diphenoxylate HCl, domperidone, famotidine, loperamide, mesalazine, nizatidine, omeprazole, ondansetron HCL, ranitidine HCl, sulphasalazine. Histamine H,-receptor antagonists, for example acrivastine, astemizole, cinnarizine, cyclizine, cyproheptadine HCl, dimenhydrinate, flunarizine HCl, loratadine, meclozine HCl, oxatomide, terfenadine. Lipid regulating agents, for example bezafibrate, clofibrate, fenofibrate, gemfibrozil, probucol.Nitrates and other anti-anginal agents, for example amyl nitrate, glyceryl trinitrate, isosorbide dinitrate, isosorbide mononitrate, pentaerythritol tetranitrate.Nutritional agents, for example betacarotene, vitamin A, vitamin B₂, vitamin D, vitamin E, vitamin K.Opioid analgesics, for example codeine, dextropropyoxyphene, diamorphine, dihydrocodeine, meptazinol, methadone, morphine, nalbuphine, pentazocine. Sex hormones: clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone.Stimulants, for example amphetamine, dexamphetamine, dexfenfluramine, fenfluramine, mazindol.

A preferred group of therapeutically active agents are those which are more effectively delivered in an encapsulated form because the distribution of the encapsulated agent in the organism after intravenous injection is more favourable with regard to efficacy or side effects than the administration of a solution of the agent. Preferred examples of such hydrophobic drugs with disadvantageous side effects are paclitaxel, docetaxel, epothilones and derivatives such as thia-epothilones, and camptothecins, which have a low solubility in water.

In an embodiment of the present invention, a primary lipid dispersion is frozen and thawed to obtain a secondary dispersion. The freezing method, temperature and freezing time may be varied. In one embodiment of the invention the suspension is frozen at a temperature of about −30° C. in an ordinary freezer. In another embodiment of the invention, the suspension is frozen in a cryogenic liquid like liquid nitrogen.

Thawing of the dispersion might be performed at different temperatures and for different periods of time. Also the temperature might be raised during the thawing process.

In another embodiment, the primary dispersion is dehydrated. Dehydration can be performed by various methods such as through liquid evaporation, solid or glass state or antisolvent/precipitation techniques. Dehydration by evaporation of the dispersion is preferably performed under reduced pressure, i.e., the dispersion may be lyophylised or freeze-dried. In another embodiment the liposomes are dehydrated by spray-drying. In another embodiment the preparation is dehydrated using supercritical or near supercritical phases.

In the embodiments of the invention wherein the primary lipid dispersion has been dehydrated, it is a further aspect that the dehydrated lipid dispersion is rehydrated in a suitable medium to obtain a second lipid dispersion. Preferably, the medium is an aqueous medium. Besides water, the aqueous medium may comprise one or more further liquid constituents which are at least partially miscible with water, preferably an organic solvent, more preferably an alcohol (e.g. a C₁₋₄ alcohol such as methanol, ethanol, propanol, butanol and combinations thereof, etc.) or a ketone (e.g. a C₁₋₄ ketone such as acetone, methylethyl-ketone and combinations thereof, etc.). Furthermore the medium may comprise additives such as, but not limited to, pH-stabilising-agents, salts, antioxidants, cryoprotectants or other excipients. Preferably, all agents or media comprised in the dispersion are pharmaceutically acceptable.

In another embodiment, the medium may comprise a further active agent.

In order to solubilise an agent in a lipid dispersion, it is an aspect of the current invention that the agent incubated with the lipid dispersion in an undissolved form, i.e. amorphous or as crystals of different size and morphology. During incubation, the dispersion may be agitated, preferably by stirring, shaking or tumbling. Also the incubation may be performed at different temperatures depending on the properties of the lipid membrane and the solubilised agent. Preferably, the incubation is performed at a temperature above the phase transition temperature of the lipids. In another preferred embodiment the incubation is performed between 2° C. and 40° C. The incubation may be performed until a maximal or desired amount of the active agent has been solubilised in the dispersion. The conditions of the incubation like temperature, incubation time, composition of the dispersion (like lipid concentration, pH, salt concentration, amount of active agent and the like) may be varied. In one embodiment, incubation is performed less than one hour. In another embodiment of the invention, incubation is performed less than about 45 minutes, preferably about 30 minutes or less. In another embodiment, incubation is performed less than 15 minutes.

In one embodiment, unsolubilsed active agent is separated from the dispersion after the incubation. In a preferred embodiment, unsolubilised agent is separated by centrifugation or filtration. Filtration might be performed in a syringe filter.

In a preferred embodiment of the current invention, all materials employed in the current invention are sterile and all devices used to practise the invention are sterile. In another embodiment, the dispersion is sterilised after the solubilisation of the active agent. Preferably, the dispersion is sterilized by sterile filtration, but any other suitable sterilization process might be applied.

It is one aspect of the current invention to disclose a lipid dispersion, preferably a liposomal preparation, obtainable or obtained by the method described herein. Such dispersion may be used as a medicament or a diagnostic for administration to a subject in need thereof, preferably to a mammal or a human patient.

It is a further embodiment that the inventive lipid dispersion can be used for the treatment or diagnosis of a disease, preferably of a disease associated with enhanced angiogenesis as for example cancer diseases, chronic inflammatory diseases, rheumatoid arthritis, dermatitis, psoriasis, neovascularisation diseases of the eye, for example age related macular degeneration or diabetic retinopathy, multiple sclerosis, wound healing and others. Examples for cancers diseases are lymphoma, melanoma, non-small-cell lung cancer, ovarian cancer, prostate cancer and to childhood cancers such as brain stem glioma, cerebellar astrocytoma, cerebral astrocytoma, ependymoma, Ewing's sarcoma/family of tumors, germ cell tumor, extracranial, Hodgkin's disease, leukaemia, acute lymphoblastic, leukaemia, acute myeloid, liver cancer, medulloblastoma, neuroblastoma, non-Hodgkin's lymphoma, osteosarcoma/malignant fibrous histiocytoma of bone, retinoblastoma, rhabdomyosarcoma, soft tissue sarcoma, supratentorial primitive neuroectodermal and pineal tumors, unusual childhood cancers, visual pathway and hypothalamic glioma, Wilms Tumor and other childhood kidney tumors and to less common cancers including acute lymphocytic leukaemia, adult acute myeloid leukaemia, adult non-Hodgkin's lymphoma, brain tumor, cervical cancer, childhood cancers, childhood sarcoma, chronic lymphocytic leukaemia, chronic myeloid leukaemia, esophageal cancer, hairy cell leukaemia, kidney cancer, liver cancer, multiple myeloma, neuroblastoma, oral cancer, pancreatic cancer, primary central nervous system lymphoma, skin cancer, small-cell lung cancer, head & neck cancer, gall bladder and bile duct cancer, stomach cancer, gastrointestinal cancer, Kaposi's sarcoma, urothelial cell carcinoma, thyroid gland carcinoma, testicular carcinoma, vaginal cancer, angiosarcoma, soft tissue sarcoma, mesothelioma and hepatocellular carcinoma.

Further to the disclosed lipid dispersion comprising an active agent the medicament or diagnostic may comprise a pharmaceutical acceptable carrier, diluent and/or adjuvant.

In a further embodiment, the invention relates to a lipid dispersion comprising an active agent, wherein less than 10%, preferably less than 6%, of the active agent is released from the lipid phase of said dispersion over at least 3 days, preferably at least 14 days. Preferably, the lipid dispersion is a liposomal preparation. Preferably, the lipid dispersion comprises DOTAP and DOPC. Preferably, the active agent is paclitaxel.

In another aspect the invention discloses a kit which comprises the material used to practice the inventive processes. The kit comprises liquid, frozen or dehydrated lipid dispersion, if suitable a rehydration buffer, and an instruction manual describing the steps of the process. The kit may also comprise a therapeutic or a diagnostic agent. The kit may also comprise a syringe and a filter.

In another aspect of the present invention a method for increasing the solubilisation efficiency of preformed liposomes for an active agent is disclosed. Said method comprises treating preformed liposomes with freezing and thawing and/or dehydrating and rehydrating prior to solubilising the active agent in said treated liposomes. The increase of solubilisation efficiency refers to the solubilised amount of a specified active agent in a pre-treated lipid dispersion as described compared to the solubilised amount of such an agent in a lipid dispersion which was not pre-treated as described. In the experimental part examples with an increase in the range of 10% to 400% are given.

It should be noted that all preferred embodiments discussed for one or several aspects of the invention also relate to all other aspects.

### DEFINITIONS

“About” in the context of amount values refers to an average deviation of maximum +/−20%, preferably +/−10% based on the indicated value. For example, an amount of about 30 mol % cationic lipid refers to 30 mol % +/−6 mol % and preferably 30 mol % +/−3 mol % cationic lipid with respect to the total lipid/amphiphile molarity.

“Active agent” refers to a compound, or mixture of compounds, having a particular bioactivity based on which it is useful as an agent useful for the diagnosis, prevention, or treatment of a human or animal disease or condition. Drug substances and diagnostic agents are important examples of active agents according to the present invention.

“Amphiphile” refers to a molecule, which consists of a water-soluble (hydrophilic) and an oil-soluble (lipophilic) part. The lipophilic part preferably contains at least one alkyl chain having at least 10, preferably at least 12 carbon atoms.

“Angiogenesis associated disease” refers to a disease which is characterized by enhanced angiogenesis as disclosed for example in McDonald et al., U.S. Pat. No. 5,837,283, or Strieth et al., 2004, Int. J. Cancer 110, 117-124.

“Aqueous medium”, “aqueous liquid” or “aqueous phase” a used herein refers to a liquid material which comprises water. The material may represent a single liquid phase, or a two- or multiphase system in which the continuous phase is liquid and comprises water. Thus, an aqueous dispersion, aqueous suspension or an emulsion in which the continuous phase is aqueous are also examples of aqueous liquids. An aqueous liquid which contains a colloidal material is hereinafter sometimes referred to as an aqueous colloidal dispersion or solution.

“Cationic” refers to an agent that has a net positive charge or positive zeta potential under the respective environmental conditions. In the present invention, it is referred to environmental conditions where the pH is in the range between 3 and 9, preferably between 5 and 8.

“Cryoprotectant” refers to a substance that helps to protect a species from the effect of freezing.

“Colloidal” refers to a size range, such as an average diameter, of about 10 nm to about 10 μm. More preferably, the colloidal particles of the invention have an average diameter of about 20 nm to about 5 μm, and particularly from about 50 nm to about 1 μm.

“Diagnostically active agent” or “diagnostic” refers to a pharmaceutically acceptable agent that can be used to visualise a biological property or state in a subject or sample by various methods. The visualisation can be used to make a diagnosis.

“Dehydration” or “dehydrate” refers to the process of withdrawing water from a composition. The water might be withdrawn from the composition to a residual content of lower than about 10% w/w, preferably lower than about 5% w/w.

“Excipient” refers to a pharmaceutically acceptable, pharmacologically substantially inert material as for example glucose, saccharose, raffinose, galactose, maltose, lactose, mannitol, sorbitol and trehalose.

“Lipid” refers to its conventional sense as a generic term encompassing fats, lipids, alcohol-ether-soluble constituents of protoplasm, which are insoluble in water. Lipids are composed of fats, fatty oils, essential oils, waxes, steroid, sterols, phospholipids, glycolipids, sulpholipids, aminolipids, chromolipids, and fatty acids. The term encompasses both naturally occurring and synthetic lipids. Preferred lipids in connection with the present invention are: steroids and sterol, particularly cholesterol, phospholipids, including phosphatidyl and phosphatidylcholines and phosphatidylethanolamines, and sphingomyelins. Where there are fatty acids, they could be about 12-24 carbon chains in length, containing up to 6 double bonds. The fatty acids are linked to the backbone, which may be derived from glycerol. The fatty acids within one lipid can be different (asymmetric), or there may be only 1 fatty acid chain present, e. g., lysolecithins. Mixed formulations are also possible, particularly when the non-cationic lipids are derived from natural sources, such as lecithins (phosphatidylcholines) purified from egg yolk, bovine heart, brain, or liver, or soybean.

“Lipid dispersion” refers to a dispersion of at least one lipid or amphiphile in an aqueous medium. Hence, the lipid dispersion of the present invention comprises an aqueous phase and a lipid phase. Examples of lipid dispersions are liposomes, micelles, emulsions, gels, (inverted) hexagonal phases, cubic phases, or any other colloidal or lyotropic phase state of amphiphiles (Evans, Wennerstrom, The Colloidal Domain: Where Physics, Chemistry Biology and Technology meet, VHC, New York, 1994, pp 131-181, 239-283, 285-323, 451-496).

“Liposomes” are artificial lipid bilayer vesicles of various sizes and structures. Unilamellar vesicles are liposomes defined by a single lipid bilayer enclosing an aqueous space. In contrast, oligo- or multilamellar vesicles comprise several membranes. Typically, the membranes are roughly 4 nm thick and are composed of amphiphilic lipids, such as phospholipids of natural or synthetic origin. Optionally, the membrane properties can be modified by the incorporation of other lipids such as sterols or cholic acid derivatives. Liposomes with particularly flexible membranes based on phospholipids with a low phase transition temperature (i.e. below body temperature) are sometimes referred to as transfersomes.

Depending on their diameter and number of bilayer membranes, liposomes may also be classified as multilamellar vesicles (MLV, two or more bilayers, typically above approx. 150 to 200 nm), small unilamellar vesicles (SUV, one single bilayer, typically below about 100 nm), multivesicular vesicles (MVV, several vesicular structures within a larger vesicle), and large unilamellar vesicles (LUV, one single bilayer, typically larger than about 100 nm).

The “log P” indicates the degree to which an agent is partitioned between water and octanol (or other non-miscible solvent). Generally, a higher Log P number means that an agent is better soluble in octanol. The log P is defined as log P=log ([concentration of the agent in octanol]/[concentration of the agent in water]).

“Negatively Charged Lipids” refer to lipids that have a negative net charge.

“Pharmaceutically acceptable” is meant to encompass any substance, which does not interfere with effectiveness of the biological activity of the active ingredient and that is not toxic to the host to which it is administered.

“Pharmaceutical composition” refers to a combination of two or more different components with superior pharmaceutical properties than are possessed by either component. In the present invention, the two or more components refer to a lipid or colloidal dispersion and an active agent, optionally together with a pharmaceutically acceptable carrier, diluent and/or adjuvant.

The “phase transition temperature” is the temperature of the transition from a liquid like (liquid crystalline) to a solid like (gel) phase of the lipid membrane. In the literature it is also called “main phase transition” or Tₘ. For lyotropic lipid phases, several other types of phase transitions can be observed.

“Phospholipid” refers to a lipid consisting of a glycerol backbone, a phosphate group and one or more fatty acids which are bound to the glycerol backbone by ester bonds.

“Poorly soluble in an aqueous medium” refers to the property of a substance of having a solubility of lower than 0.1 mg/ml, preferably lower than 0.05 mg/ml, most preferably lower than 0.01 mg/ml in water at physiological pH at room temperature.

“Positively Charged Lipids” refer to a synonym for cationic lipids (for definition see definition of “cationic lipids”). In the present invention, it is referred to environments where the pH is in the range between 3 and 9, preferably between 5 and 8.

“Small molecule” refers to an organic agent with a molecular weight of about 1000 Da or less.

“Solubilising” refers to the transfer of a substance into the aqueous or lipid phase of a dispersed system. In an embodiment where a substance is solubilised in a liposomal suspension, the substance may be present in the aqueous phase in a hydrated state or may be bound or integrated into the lipid phase of the liposomal membranes.

“Solubilisation efficacy” and “solubilisation capacity” as used in the present invention refer to the molar amount of an agent which is solubilised in a lipid dispersion as disclosed per molar amount of lipid comprised in the lipid dispersion. A higher amount of an agent solubilised by a certain amount of lipid reflects a higher loading efficacy and thereby a higher solubilisation efficiency of the respective lipid dispersion.

“Therapeutically active agent” or “therapeutic agent” refers to an agent which prevents or reduces the extent a pathologic condition in an animal, particularly in a mammal, preferably in humans.

“Zeta potential” refers to measured electrical potential of a colloidal particle in aqueous environment, measured with an instrument such as a Zetasizer 3000 using Laser Doppler micro-electrophoresis in about 0.05 mM KCl solution at about pH 7.5. The zeta potential describes the potential at the boundary between bulk solution and the region of hydrodynamic shear or diffuse layer. The term is synonymous with “electrokinetic potential” because it is the potential of the particles which acts outwardly and is responsible for the particle's electrokinetic behaviour.

In light of the foregoing general discussion, the specific figures and examples presented below are illustrative only and are not intended to limit the scope of the invention. Other generic and specific configurations will be apparent to those persons skilled in the art.

### FIGURE LEGENDS

FIG. 1: Solubilisation of Paclitaxel by DOTAP/DOPC liposome formulations with different lipid concentrations. Results for a liquid formulation (triangles) in comparison to a formulation after freezing and thawing (diamonds) are shown. Experimental procedures are described in the general procedures part.

FIG. 2: Solubilisation of Paclitaxel by liposome formulations with different molar ratios of DOTAP and DOPC. The liposomes had a total lipid concentration of 10 mM. Treated and untreated liposomes are compared.

FIG. 3: Comparison of formulations composed of lipids in different phase states.

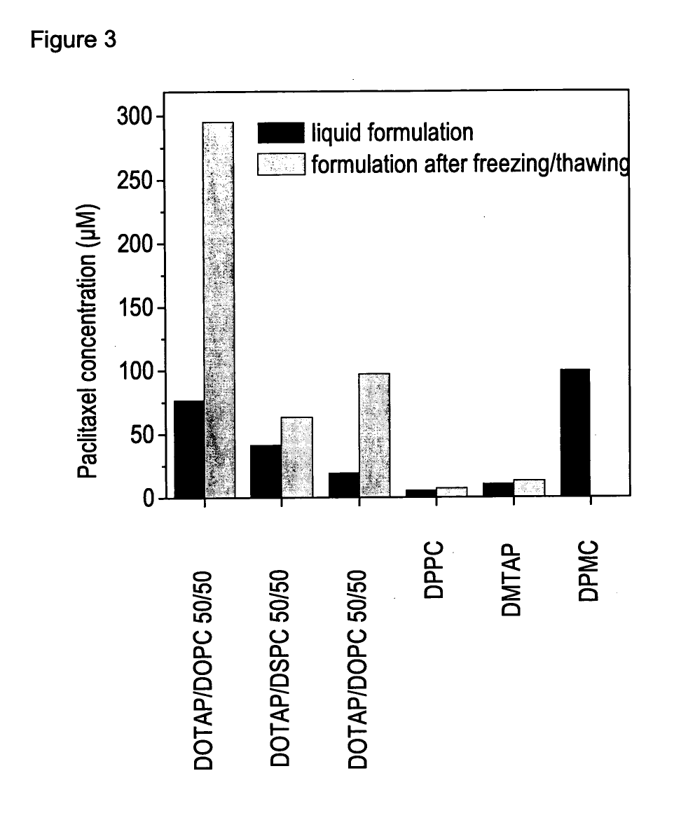

FIG. 4: Influence of excipients on the solubilisation capacity of DOTAP/DOPC liposomes.

FIG. 5: Comparison of solubilisation capacity of DOTAP/DOPC liposomes as untreated liquid formulation and after different types of pre-treatment.

FIG. 6: Amount of Paclitaxel solubilised by freeze-dried or liquid formulation DOTAP/DOPC liposomes (10 mM) for different incubation periods from 15 minutes to three hours.

FIG. 7: Stability of a preformed formulation comprising Paclitaxel as a function of time after preparation. One part of the formulation was tested without further treatment (squares, liquid formulation) and the other part was treated by a freeze/thaw cycle (circles, formulation after freeze/thawing)

### EXAMPLE

### 1. General Procedures

### 1.1 Materials

Paclitaxel was obtained from Cedarburg Pharmaceuticals (Grafton, Wis., USA), Camptothecin from Boehringer Ingelheim (Ingelheim, Germany). Both substances were used without further purification. All lipids were purchased from Avanti Polar Lipids (Alabaster, Ala., USA). Trehalose-Dihydrate was obtained from Ferro Pfanstiehl (Cleveland, Ohio, USA), all other excipients were from Merck (Darmstadt, Germany). Solvents were obtained from Merck (Darmstadt, Germany) and were of analytical or HPLC grade. Syringe filters Minisart NML (Sartorius, Göttingen, Germany) of 0.2 μm pore size from Millipore were used to remove excess drug.

### 1.2. Methods

### 1.3. Preparation of Empty Liposomes

Formulations of empty liposomes were prepared by film method or ethanol injection. Unless otherwise noted in the examples all formulations were made at a concentration of 10 mM total lipid in a solution of 10% (w/w) trehalose in water.

1.4. Film Method

A solution of the required amounts of lipids in chloroform was added to a round bottom flask. The solvent was evaporated to dryness in a rotary evaporator (Heidolph, Germany). Subsequently the dry film was hydrated in water or a solution of excipient(s) in water to the required lipid concentration by gently shaking the flask. The resulting suspension of multilamellar liposomes was extruded five times through a polycarbonate membrane of a pore size of 200 nm at a pressure of about 5 bar.

### 1.5. Ethanol Injection

A 400 mM stock solution of lipids in ethanol was prepared. The required volume of the stock solution to provide a final lipid concentration of 10 mM was injected under stirring into water or a solution of excipient(s) in water. The resulting suspension was stirred for another 15 minutes and subsequently extruded 5 times through a polycarbonate membrane of a pore size of 200 nm at a pressure of about 5 bar.

### 1.6. Freeze/Thaw Treatment

The formulations were stored in a freezer at −30° C. for at least one day and subsequently thawed at room temperature.

### 1.7. Freeze-Drying Treatment

Liposomes prepared by film method or ethanol injection as described above were freeze-dried. An example of suitable process parameters is as follows:

| | | Temperature | Time | Pressure |
|---|---|---|---|---|
| Step | Process step | (° C.) | (h:min) | (mbar) |
| 1 | preparation | +4 | 1:00 | 1000 |
| 2 | freezing | −40 | 0:22 | 1000 |
| 3 | freezing | −40 | 4:45 | 1000 |
| 4 | preparation | −40 | 0:15 | 1000 |
| 5 | primary drying | −40 | 0:01 | 0.1 |
| 6 | primary drying | −16 | 3:00 | 0.1 |
| 7 | primary drying | −16 | 90:00 | 0.1 |
| 8 | secondary drying | +20 | 3:00 | 0.01 |
| 9 | secondary drying | +20 | 12:00 | 0.o1 |

1.8. Incubation of Liposomes with Paclitaxel and Separation of Unsolubilised Drug

A dispersion of empty liposomes was prepared as described above and added to dry Paclitaxel. If not specified otherwise, the mixture was stirred for three hours at room temperature. Subsequently unsolubilised Paclitaxel was separated by filtration or centrifugation. Filtration was performed with a syringe filter of 0.2 μm pore size.

### 1.9. Quantification of Paclitaxel

The concentration of solubilised Paclitaxel was determined by means of HPLC. After separation from the unsolubilised drug by filtration of the liposome preparation through a filter of 200 nm pore size, the samples were diluted in suitable solvent mixtures, typically the mobile phase for the HPLC analysis. The Agilent 1100 Series HPLC System (Agilent Technologies, Palo Alto, Calif., USA) consisted of a quaternary pump, an autosampler, a column thermostat and an on-line degasser. A LiChrospher 60 RP (5 μm, 4×250 mm) analytical column (Merck, Darmstadt, Germany) was used. The flow rate of the mobile phase, consisting of 32% (v/v) acetonitrile, 12% tetrahydrofuran and 56% ammoniumacetat (2 mM in water, pH value set to 4.8 with acetic acid) was set at 1 ml/min.

The run time was 40 minutes. The detection was performed with a variable wavelength detector at 229 nm.

### 2. Experiments

2.1. Drug Loading into Treated and Untreated Formulations

With this example, the fundamental principle of drug loading into empty liposomes and the improvement of drug loading capacity by pre-treatments is demonstrated.

Liposomes composed of DOTAP/DOPC (50/50 mol/mol) with a total lipid concentration in the range of from 10 mM to 35 mM in an aqueous solution of 10% trehalose were prepared by the film method as described in the general procedures part. Freshly prepared liposomes (liquid formulation) and liposomes which had been treated subsequently by freezing/thawing (formulation after freezing/thawing) (see 1.6) were investigated with respect to their capacity for solubilising Paclitaxel. Both liposome preparations were exposed to Paclitaxel by adding dry Paclitaxel crystals as provided by the manufacturer to the liposome suspension and gently stirred for 3 hours. Subsequently the preparations were filtrated through a filter of 200 nm pore size to remove the excess (unsolubilised) drug. The amount of solubilised Paclitaxel in the filtrate was determined by HPLC analysis as described under Experiment 1.9.

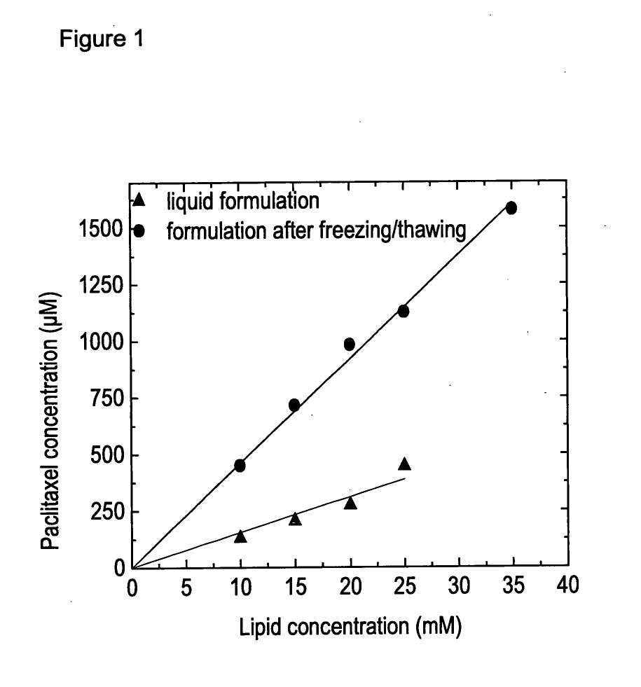

The results of the experiment as depicted in FIG. 1 show that the amount of solubilised Paclitaxel was directly proportional to the lipid concentration, i.e., the concentration of Paclitaxel in the liposomal suspension can be given as a function of the lipid concentration as:

c_{PXL}=f·cₗᵢₚ
c_{PXL}=concentration of Paclitaxel
cₗᵢₚ=lipid concentration

This demonstrates that the drug, Paclitaxel, was solubilised by the liposomes, and it can be excluded that Paclitaxel found in the aqueous filtrate was due to any artefact or boundary condition related to a particular experimental setup. In the present case, for untreated DOTAP/DOPC liposomes the solubilisation factor f was about 10⁻², i.e, 1 mM of DOTAP/DOPC resulted in a solubilised concentration of Paclitaxel of 10 μM. The solubilisation capacity of the liposomes, which had been subject to the freeze/thaw pre-treatment, was as well a linear function of the lipid concentration, however, the amount of solubilised Paclitaxel at a given lipid concentration was by about a factor of four higher, i.e., the solubilisation factor f, was about 4×10⁻². In the present example, a concentration of solubilised Paclitaxel of up to 1600 μM was obtained. No indication for an upper limit of the lipid concentration was found, i.e., the expected amount of solubilised Paclitaxel for higher lipid concentration is directly given by the solubilisation factor. For example, for liposome preparations of 100 mM concentration the expected amount of solubilised Paclitaxel would be 1 mM for the untreated and 4 mM for the treated formulation. Because in that case physical separation of solubilised drug and dispersed unsolubilised drug is more difficult, these results have not been used for the current analysis.

Since a rather good linear dependence of the concentration of solubilised Paclitaxel as a function of lipid concentration was found, in most subsequent experiments the solubilisation factor was determined at only one fixed lipid concentration.

2.2. Variation of Lipid Composition with Identical Hydrocarbon Chains

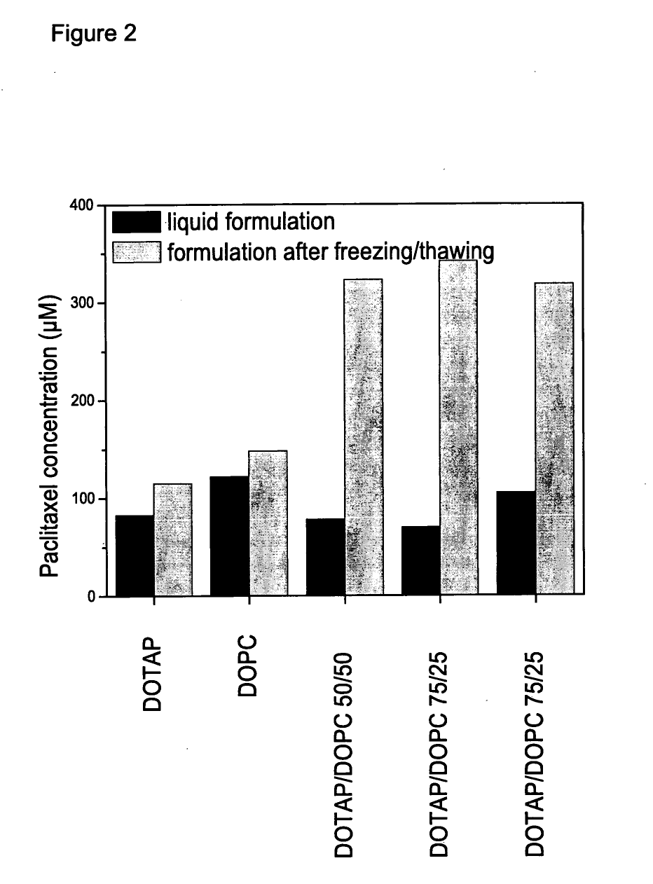

In this example the effect of lipid composition on the improvement of solubilisation capacity by freezing/thawing is demonstrated. Liposomes composed of 100% DOTAP and 100% DOPC as well as liposomes composed of DOTAP/DOPC mixtures of different molar ratios (25/75, 50/50 75/25 mol/mol) were investigated with respect to their solubilisation capacity for Paclitaxel. Both lipids, DOTAP and DOPC, have the same hydrocarbon backbone, consisting of unsaturated fatty acids. The lipids are in the liquid crystalline (fluid-like) state at room temperature. Thus, in all experiments the hydrophobic core of the lipid bilayers was the same, and only the relative fractions of the -PC and -TAP headgroups varied. Untreated and freeze/thawed pre-treated formulations (manufactured by the film method according to the general procedures) formulations with a lipid concentration of 10 mM were investigated in accordance to the procedures described in Experiment 2.1. The results are shown in FIG. 2. For DOPC a slightly higher Paclitaxel loading capacity was found compared to DOTAP, and the loading capacity of the binary mixtures reflected the molar ratio of the two components. Freezing/thawing improved the loading capacity of both pure substances, and as in the untreated case, DOPC solubilised slightly more drug than DOTAP. Remarkably, the loading capacity of liposomes from the binary mixtures of DOTAP/DOPC improved much more after freeze/thawing compared to liposomes which contained only a single lipid species. For all tested lipid mixtures, the loading capacity of the treated lipid dispersions was about a factor of four higher than in the untreated lipid dispersions.

2.3. Liposomes Composed of Lipids with Different Membrane Fluidity

Liposomes composed of lipids with different fluidity, hydrocarbon and headgroup moieties, and binary mixtures thereof, were tested for their loading capacity as described before. DOTAP/DOPC, DOTAP/DSPC, DSTAP/DSPC (all in a 50/50 molar ratio) and DPPC, DMTAP, and DPMC liposomes were prepared by the film method. The loading capacity of the untreated pure lipid liposomes varied substantially, depending on the molecular properties of the lipids. As a general rule, with increasing chain length and in the gel phase, the loading was lower. The loading capacity of all binary mixtures was substantially improved by freeze/thawing. Best improvement of the loading capacity of untreated versus treated liposomes was by a factor of 5 for the DOTAP/DSPC system.

### 2.4. Variation of Excipients

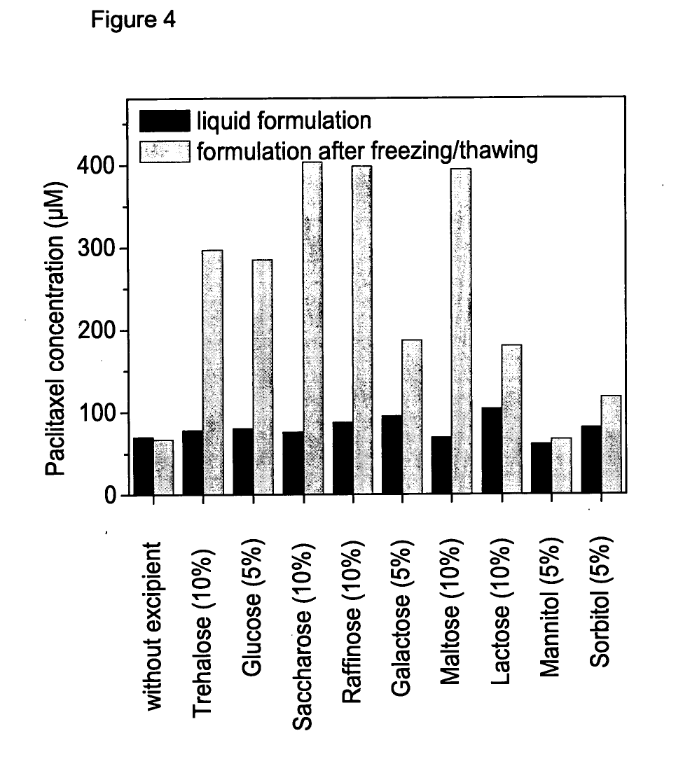

The influence of different excipients on the solubilisation capacity of liposomes was investigated. Formulations of DOTAP/DOPC 50/50 mol/mol were prepared by the film method. The lipid films were rehydrated in water and solutions of several excipients in water (10% w/w trehalose, or 5% w/w glucose, or 10% w/w saccharose, or 10% w/w raffinose, or 5% w/w galactose, or 10% w/w maltose, or 10% w/w maltose, or 5% w/w mannitol, or 5% w/w sorbitol) as described in Experiment 1.4. Liquid formulations and formulations after freezing/thawing were exposed to Paclitaxel as described before. The results are summarised in FIG. 4. Formulations without pre-treatment showed only slight differences in the solubilization in dependence on several excipients. The presence of at least one excipient was essential to improve the solubility after pre-treatment.

### 2.5. Variation of the Pre-Treatment Procedure

The invention is not limited to a particular pre-treatment method. As an example, spray dried and spray freeze-dried formulations of DOTAP/DOPC (50/50 mol/mol, 10 mM) in a solution of trehalose were loaded with Paclitaxel.

Spray drying was performed under nitrogen atmosphere (inert spray drying) using a B290 mini spray dryer (Buche, Switzerland) combined with a dehumidifier LT mini (Much, Germany) and an inert loop system B-295 (Buchi Switzerland). The liquid feed contained 10% (w/w) of trehalose, 20% ethanol (w/v) and 10 mM lipid. The liquid feed rate was 20 mL/min and the outlet temperature of the drying gas was 110° C.

For spray freeze drying, the liquid formulation was sprayed into a reservoir of liquid nitrogen. The frozen particles were transferred into suitable containers and stored into a −80 ° C. freezer until the liquid nitrogen was completely evaporated. Subsequently the frozen samples were placed into the freeze dryer on precooled shelves. Freeze drying treatment was identical to the standard freeze drying protocol as described in section 1.7. Freezing (−30° C.) was performed as described under 1.6.

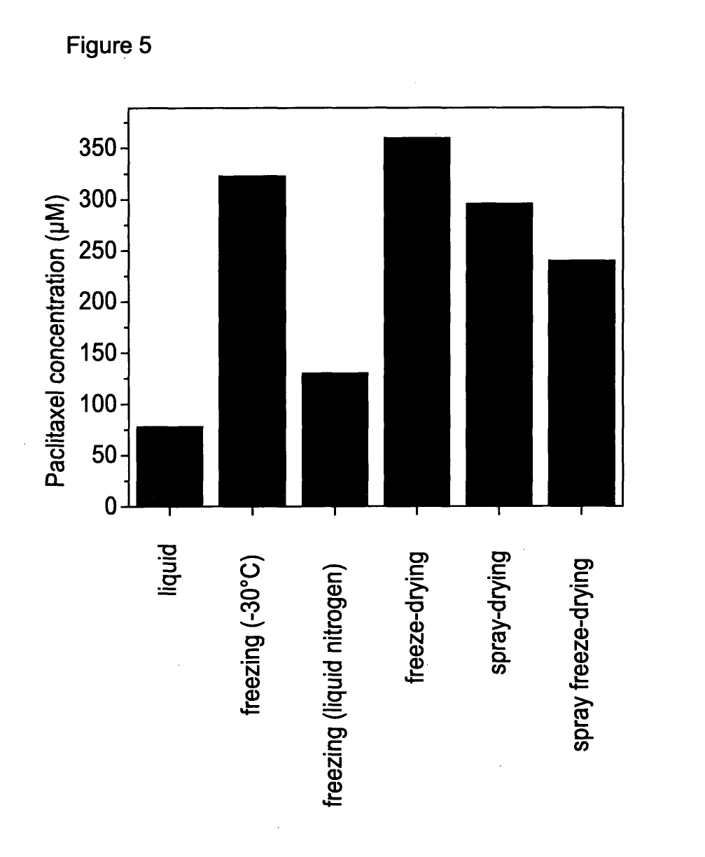

The results in FIG. 5 show that by all tested protocols the solubility of

Paclitaxel in liposomal preparations was elevated with reference to a liquid formulation without pre-treatment.

### 2.6. Loading of Different Agents

In this example Camptothecin was applied as poorly water soluble agent.

Liposomes composed of DOTAP/DOPC (50/50 mol/mol) at a total lipid concentration of 10 mM in a solution of 10% (w/w) trehalose in water were prepared according to the general procedures. Treated and untreated liposomal formulations were exposed to Camptothecin as described for Paclitaxel in Example 2.1. The concentration of solubilized Camptothecin was determined by UV/Vis-spectroscopy (Beckmann DU 640, Beckmann Coulter, Germany) at 369 nm after dilution in methanol.

The concentration of the solubilized agent could be enhanced by a factor of about three for the pre-treated formulation (52 μM) in comparison to the untreated formulation (18 μM).

### 2.7. Time Dependence of Loading

In this example time dependency of drug loading was investigated. The aim was to investigate, if a minimum time of exposure of an agent to the liposomes is required for efficient loading.

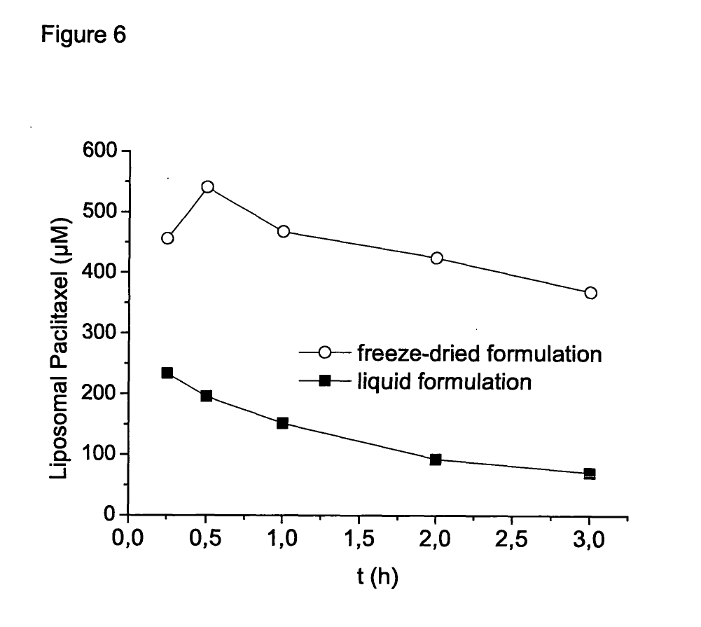

Freeze-dried liposomes composed of DOTAP/DOPC (50/50 mol/mol) were used after rehydration with water. The formulation was exposed to Paclitaxel as described above for time periods from 15 minutes to 3 hours prior to separation from the unsolubilised drug (see section 1.8). As shown in FIG. 6, even at the shortest incubation time of 15 min, the loading was not lower than in the case of 3 hour incubation.

These results suggest that an incubation time of 15 minutes is sufficient for efficient loading. Even shorter incubation times may be possible if suitable experimental procedures are applied.

### 2.8. Stability of Treated and Untreated Preparations

This example demonstrates that the inventive process is also suitable to improve stability of liposomes loaded with a drug.

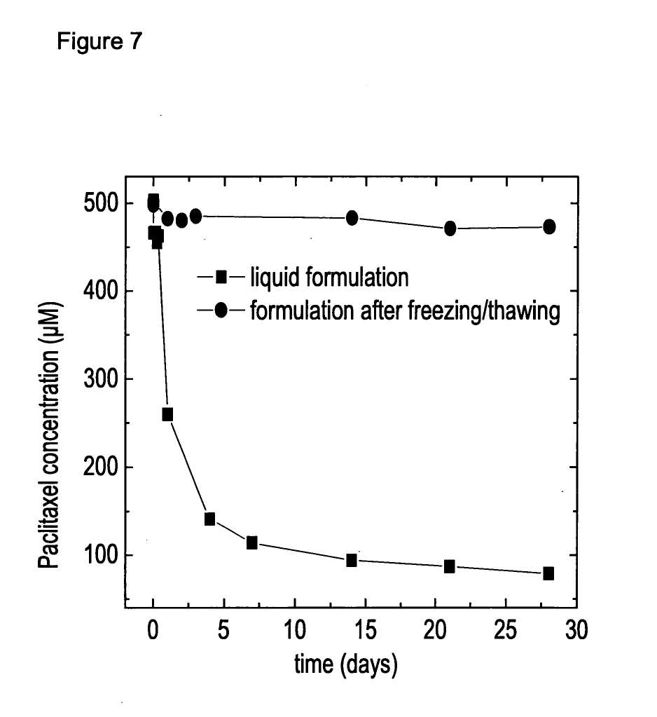

The stability of an untreated formulation was investigated in comparison to a formulation after freezing and thawing. A preformed formulation composed of DOTAP/DOPC/PXL (50/45/5, total concentration 10 mM) in a solution of trehalose in water was prepared by the film method as described in the general procedures. Subsequently, one half of the preparation was subjected to a freeze/thaw treatment as described above, the other half was left untreated. Both preparations were loaded with Paclitaxel as described and stored at room temperature at about 23° C. After specific time intervals the formulations were filtered through a 0.2 μm membrane in order to separate free Paclitaxel from liposome entrapped Paclitaxel. Subsequently, the Paclitaxel concentration in the filtered liposome preparations was determined by HPLC. The results as depicted in FIG. 7 show that only in the treated liposome preparations the Paclitaxel concentration was maintained, while in the non-treated preparation, the drug concentration dropped immediately after formation to a much lower level.

Hence, the invention permits improvement of the physical stability of a lipid dispersion comprising an active agent which was pre-treated prior to loading with the agent.

1. A process for solubilising at least one active agent in a lipid dispersion comprising incubating an active agent in an undissolved form with a lipid dispersion.

2. A process for solubilising at least one active agent in a lipid dispersion comprising the steps of:
i) freezing or dehydrating a primary lipid dispersion comprising an aqueous medium and optionally one or more excipients,
ii) thawing the frozen lipid dispersion or rehydrating the dehydrated lipid dispersion of step i) to obtain a secondary lipid dispersion,
iii) incubating the secondary lipid dispersion of step ii) with an active agent.

3. A process according to claim 2, wherein the active agent of step iii) is present in an undissolved form.

4. A process according to claim 1, wherein no freezing or dehydrating step is performed after incubating said active agent with said lipid dispersion.

5. A process according to claim 1, wherein the active agent is hydrophobic and/or has a low solubility in water.

6. A process according to claim 1, wherein partitioning of said active agent between an aqueous phase and a lipid phase is predominantly in the lipid phase.

7. A process according to claim 1, wherein said active agent which is present in an undissolved form is in an amorphous or crystalline form.

8. A process according to claim 1, wherein the lipids comprised in the lipid dispersion have a phase transition temperature which is lower than room temperature (23° C.).

9. A process according to claim 1, wherein said lipid dispersion comprises at least one, preferably two different types of lipids.

10. A process according to claim 1, wherein said lipid dispersion comprises two different types of lipids in a ratio between about 90:10 and 10:90, more preferably in a ratio of or between about 75:25 and 25:75.

11. A process according to claim 1, wherein the lipid dispersion comprises DOTAP and DOPC.

12. A process according to claim 1, wherein at least one lipid of said lipid dispersion comprises at least one unsaturated or branched alkyl chain.

13. A process according to claim 1, wherein said lipid dispersion is a colloidal dispersion, preferably a liposomal suspension.

14. A process according to claim 1, wherein said active agent is a therapeutically and/or diagnostically active agent.

15. A process according to claim 14, wherein the active agent is a small molecule.

16. A process according to claim 2, wherein the excipient is selected from the group comprising water-soluble sugars selected from the group consisting of glucose, saccharose, raffinose, galactose, maltose, lactose, mannitol, sorbitol or trehalose.

17. A process according to claim 16, wherein the excipient is trehalose.

18. A process according to claim 1, wherein incubating said active agent with said lipid dispersion is performed in less than about 3 hours, preferably in less than about 1.5 hours, more preferably in less than about 60 minutes and most preferably in less than about 30 minutes.

19. A process according to claim 1, wherein a separation step is performed subsequently to incubating the undissolved active agent with the lipid dispersion, wherein unsolubilised active agent is removed.

20. A process according to claim 19, wherein said separation step is performed by filtration or centrifugation.

21. A lipid dispersion comprising at least one active agent obtainable by the process of claim 1.

22. A lipid dispersion comprising an aqueous medium and an active agent, wherein less than about 6%, of the active agent is released into the aqueous medium of said dispersion in at least 3 days.

23. A lipid dispersion according to claim 21, wherein the lipid dispersion is a liposomal preparation.

24. A lipid dispersion according to claim 21, wherein the lipid dispersion comprises DOTAP and DOPC.

25. A lipid dispersion according to claim 21, wherein said active agent is paclitaxel.

26. A lipid dispersion according to claim 21, wherein the active agent is a therapeutically active agent, and wherein the lipid dispersion optionally comprises a pharmaceutical acceptable carrier, diluent and/or adjuvant, for use as a medicament.

27. A lipid dispersion according to claim 21, wherein said active agent is a diagnostically active agent, for use as a diagnostic.

28. A method of treating or diagnosing a disease by administering a dispersion comprising at least one active agent obtainable by the process of claim 1 to a subject in need thereof, preferably to a human patient.

29. A kit comprising a frozen or dehydrated lipid dispersion, optionally a rehydration buffer, an instruction manual and optionally a diagnostic or therapeutic agent.

30. A method for increasing the solubilisation efficiency of a lipid dispersion for an active agent comprising treating said lipid dispersion with freezing and thawing and/or dehydrating and rehydrating prior to adding said active agent.

31. A method according to claim 30, wherein the solubilisation efficacy is increased by a factor of at least about 10% to at least about 100%.

32. A method according to claim 30 or 31, wherein the increased solubilisation efficacy of the lipid dispersion is maintained for at least 7 days.
